(19) United States
(12) Patent Application Publication
(10) Pub. No.: US 2023/0248374 A1
(43) Pub. Date: Aug. 10, 2023

(54) SYSTEM AND METHOD TO ALIGN AN IMPLANT KEEL PUNCH

(71) Applicant: THINK SURGICAL, INC., Fremont, CA (US)

(72) Inventors: Micah Forstein, Fremont, CA (US); Mark Dixon, Fremont, CA (US)

(73) Assignee: THINK SURGICAL, INC., Fremont, CA (US)

(21) Appl. No.: 18/012,732

(22) PCT Filed: Jun. 29, 2021
(86) PCT No.: PCT/US2021/039481

§ 371 Date: Dec. 23, 2022

Related U.S. Application Data
(60) Provisional application No. 63/045,238, filed on Jun. 29, 2020.

(51) Int. Cl. A61B 17/17

(52) U.S. Cl. CPC A61B 17/1764

(57) ABSTRACT

A system and method are provided for aligning a keel punch in a planned position and orientation relative to a subjects bone in a time efficient manner, which forms keel receiving features in the subjects bone with the accuracy and precision of a surgical robot. The system and method provided removes prior surgical constraints and allows a surgeon to reposition the patient prior to punching the keel receiving features without losing the alignment, and protects the robot from the forces required to punch the keel features manually, which would otherwise occur if the surgical robot were to fixedly hold the keel punching tool in place while punching the keel features. The provided method does not require a tool change, or require an end-effector to be fixedly attached to any of the components while aligning the keel punch alignment guide on the subjects bone.

### CROSS REFERENCE TO RELATED APPLICATIONS

This application claims priority benefit of U.S. Provisional Application Ser. No. 63/045,238 filed 29 Jun. 2020, the contents of which are hereby incorporated by reference.

### TECHNICAL FIELD

The present invention generally relates to an alignment system, and more particularly to a robotic surgical system and method to assist in aligning an implant keel punch during a joint replacement procedure.

### BACKGROUND

Throughout a lifetime, bones and joints become damaged and worn through normal use, disease, and traumatic events. Arthritis is a leading cause of joint damage that over time leads to cartilage degradation, pain, stiffness, and bone loss. Arthritis can also cause the muscles articulating the joints to lose strength and become painful.

If the pain associated with the dysfunctional joint is not alleviated by less-invasive therapies, a joint arthroplasty procedure is considered as a treatment. Joint arthroplasty is an orthopedic procedure in which an arthritic or dysfunctional joint surface is replaced with an orthopedic prosthesis.

The accurate placement and alignment of an implant is a large factor in determining the success of joint arthroplasty. A slight misalignment may result in poor wear characteristics, reduced functionality, poor clinical outcomes, and decreased prosthetic longevity.

Computer-assisted orthopedic surgery is an expanding field having applications in total joint arthroplasty (TJA), bone fracture repair, maxillofacial reconstruction, and spinal reconstruction. Robotic surgical systems are particularly useful for surgical procedures requiring dexterity, precision, and accuracy. For example, the TSolution One® Surgical System (THINK Surgical, Inc. Fremont, Calif.) as shown in FIG. 1, aids in the planning and execution of total joint arthroplasty procedures illustratively including total hip arthroplasty (THA) and total knee arthroplasty (TKA). The TSolution One® pre-operative planning software permits a user to pre-operatively plan the position and orientation (POSE) of a chosen bone implant (e.g., hip or knee implants) relative to three-dimensional (3-D) bone models of the patient. In the operating room, the surgical plan is transferred to the surgical robot 100 to precisely mill the bone to receive the implant as planned by the surgeon. The surgical robot 100 generally includes a base 102, a manipulator arm 104 attached to the base, and an end-effector 106 which is actuated or controlled by the manipulator arm 104 as instructed by the surgical plan. The manipulator arm 104 includes various links, joints, and sensors (e.g., encoders) to accurately actuate the end-effector 106, where the sensors can further provide feedback as to the exact position of the end-effector 106 in space. The end-effector 106 may be, for example, a tool having a tool tip 108, such as a burr or end mill cutter. The surgical robot 100 may further include a mechanical digitizer arm 110 for registering the bone, a monitor 112 to display a graphical user interface to provide workflow instructions to the user, as well as input mechanisms (not shown) for the user to interact with surgical robot 100.

In any TKA procedure, the femur and tibia are prepared to receive a femoral implant component and a tibial implant component. FIG. 2A illustrates an example of a prepared tibia T and a tibial implant component 114. The tibial implant component 114 generally includes a base plate 116 and a keel 118, where the keel 118 includes a keel post 120 and keel wings 122 to provide positional and rotational stability to the implant 114. The prepared tibia T has keel receiving features such as a post hole 124 and grooves/channels 126 to receive the keel post 120 and keel wings 122, respectively. In conventional TKA procedures, the post hole 124 and grooves 126 are formed with a keel punching tool 128 as shown in FIG. 2B. The keel punching tool 128 generally includes a handle 130 and a keel punch 132. The keel punch 132 is designed with features matching those of the keel 118 and may therefore have a keel post punch component 134 and a keel groove punch component 136. After the tibia T is resurfaced, a user aligns the keel punching tool 128 and punches the keel punch 132 into the tibia T to form the post hole 124 and channels 126. The tibial implant component 114 is then implanted on the tibia T.

In a robotic-assisted TKA procedure, the surgical robot may mill the post hole 124 and channels 126 directly in the tibia T and is more accurate in doing so compared to conventional procedures. However, current patient positioning and robot workspace constraints may limit this ability. For example, FIG. 3 depicts one situation where the surgical robot 100 may have difficulty forming the channels 126 in the tibia T. The end-effector 106 of the robot 100 may include in certain inventive embodiments, a housing 140, a motor within the housing, and a tool driven by the motor where the tool extends from the housing 140. The tool may include in certain inventive embodiments, a shaft and tool tip 108, where the shaft of the tool is disposed within a sleeve 142. As the surgical robot 100 attempts to mill the channels 126 in the tibia T, the position of the femur F may interfere or collide with the sleeve 142 and prohibit the formation of the channels 126. In this case, a user may need to reposition the femur F or tibia T to provide enough clearance for the end-effector 106, which can increase the overall surgical time of the procedure. For surgical systems that require the bone to be fixed relative to the surgical robot, the repositioning for the femur F or tibia T is particularly laborious as the bones need to be unfixed, re-positioned, re-fixed, and then re-registered.

In addition to patient positioning and workspace constraints, the formation of the channels 126 may require a tool change to a tool having a smaller diameter tool tip 108. The tool change may likewise increase the overall surgical time of the procedure. Therefore, in some instances, the surgical robot may be configured to prepare the entirety of the bone (i.e., resurface the tibial plateau), and then mill small alignment features in the tibia to assist a surgeon with aligning a conventional keel punching tool to then form the keel receiving features. While this technique is quite accurate, small alignment errors can occur especially in the internal-external rotational degree-of-freedom.

Thus, there exists a need for a system and method to accurately align a keel punching tool in a planned position and orientation relative to a bone in a time efficient manner There is a further need to form one or more keel receiving features in a bone with the accuracy and precision of a surgical robot.

### SUMMARY

A system is provided for aligning a keel punch in a planned position and orientation to form one or more keel receiving features in a material. The system includes a keel punch, a keel punch alignment guide, a keel post tool, and a set of securements. The keel punch alignment guide is configured to guide the keel punch to form the one or more keel receiving features in the subject's bone. The keel post tool is configured to be temporarily inserted into a prepared post hole formed in the material to assist with aligning the keel punch alignment guide. A securement can be employed that is configured to be inserted through apertures in the keel punch alignment guide and into the material.

A method is provided for aligning a keel punch in a planned position and orientation relative to a bone of a subject to form one or more keel receiving features in the bone during a computer-assisted surgical procedure. The method includes positioning an end-effector of a surgical robot to an alignment position based on at least one of a geometry of a keel punch alignment guide, a geometry of an implant component, and a planned position and orientation (POSE) for the implant component relative to the bone. A keel punch alignment guide is then placed on a prepared bone surface of the bone, and an end-effector interaction member is aligned on the keel punch alignment guide with the end-effector of the surgical robot. The aligned keel punch alignment guide is then secured to the bone, and the end-effector s removed.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 depicts a prior art surgical robot configured to assist with total joint replacement procedures;

FIG. 2A depicts a prior art view of a proximal tibia prepared to receive a tibial implant component;

FIG. 2B depicts a prior art keel punching tool to form keel receiving features in a tibia;

FIG. 3 illustrates an example of a potential problem that may occur when an end-effector of a surgical robot attempts to form keel receiving features in a tibia;

FIGS. 4A and 4B depict a keel punch alignment guide in accordance with embodiments of the invention, where FIG. 4A depicts a top perspective view thereof, and FIG. 4B depicts a bottom perspective view thereof;

FIG. 5 depicts a temporary keel post to assist with aligning the keel punch alignment guide in accordance with embodiments of the invention;

FIGS. 6A to 6E illustrate a series of method steps to form keel receiving features in a tibia in a planned location in accordance with embodiments of the invention, where FIG. 6A depicts a keel post hole formed in a tibia, FIG. 6B depicts the keel punch alignment guide assembled on the tibia, FIG. 6C depicts the keel punch alignment guide and temporary keel post assembled on the tibia with an end-effector aligning the keel punch alignment guide in internal-external rotation, FIG. 6D depicts the keel punch alignment guide being fixed to the tibia in the aligned position, and FIG. 6E depicts the keel punch alignment guide fixed to the tibia and ready to guide a keel punching tool into the tibia;

FIGS. 7A-7C depict a method for defining an alignment position for an end-effector to assist in aligning a keel punch alignment guide on a bone in accordance with embodiments of the invention, where FIG. 7A depicts a tibial implant model having an axis and a point defined relative to the tibial implant model, FIGS. 7B depicts an implant model in a planned position and orientation (POSE) relative to a bone model, and FIG. 7C depicts the registration of the bone model to a tibia bone in the operating room; and

FIG. 8 depicts a robotic surgical system to assist in the alignment of a keel punch alignment guide on a bone in accordance with embodiments of the invention.

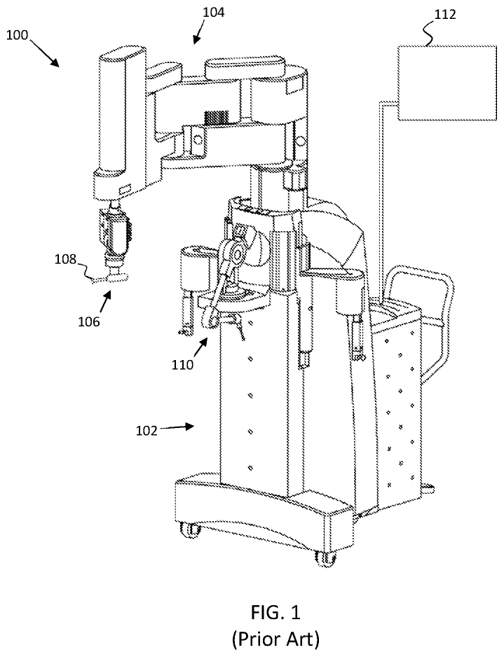

### DETAILED DESCRIPTION

The present invention has utility as an improved system and method for accurately aligning a keel punch in a planned position and orientation in a material. While the present invention is further detailed with respect to bone alignment as part of an anatomical joint replacement procedure, it is appreciated that the present invention also finding utility in the precise joining of mechanical components formed from a variety of materials such as metals, composites, ceramics, and combinations thereof. Anatomical joints that benefit from the present invention illustratively include a knee, an elbow, a hip, a finger, a toe, a wrist, an ankle, a mandible, and an inter-vertebral interface The present invention is particularly useful for forming keel receiving features in a bone with the accuracy and precision of a surgical robot. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “pre-operative bone data” refers to bone data used to pre-operatively plan a procedure before making modifications to the actual bone. The pre-operative bone data may include in certain inventive embodiments, one or more of the following. An image data set of a bone (e.g., computed tomography, magnetic resonance imaging, ultrasound, x-ray, laser scan), a virtual generic bone model, a physical bone model, a virtual patient-specific bone model generated from an image data set of a bone, or a set of data collected directly on a bone intra-operatively commonly used with imageless computer-assist devices.

As used herein, the term “digitizer” refers to a device capable of measuring, collecting, designating, or recording the position of physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described in, for example, U.S. Pat. No. 7,043,961; a digitizer probe as described in U.S. Pat. No. 8,615,286; or an end-effector of a robotic device.

As used herein, the term “digitizing” refers to the collecting, measuring, designating, and/or recording of physical points in space with a digitizer.

Also described herein are “robotic surgical systems.” A robotic surgical system refers to any system requiring a robot to aid in a surgical procedure. Examples of a robot surgical system include 1-N degree of freedom hand-held surgical system, automatic or semi-automatic serial-chain manipulator systems, haptic serial chain manipulator systems, parallel robotic systems, or master-slave robotic systems, as described in U.S. Pat. Nos. 5,086,401; 7,206,626; 8,876,830; 8,961,536; and 9,707,043; and U.S. Patent Publications 2018/0344409 and 2019/0388099. In particular inventive embodiments, the surgical system is a robotic surgical system as described below. In another embodiment, the robot surgical system is a haptically controlled system where a user freely wields the end-effector to mill the bone while the system haptically constrains the end-effector within an envelope or boundary as defined in a surgical plan. The surgical system may provide automatic control, semi-automatic control, power control, haptic control, or any combination thereof.

Also, referenced herein is a surgical plan. For context, a surgical plan is created, either pre-operatively or intra-operatively, by a user using planning software. The planning software may be used to plan the position for an implant relative to pre-operative bone data. For example, the planning software may be used to generate three-dimensional (3-D) models of the patient's bony anatomy from a computed tomography (CT), magnetic resonance imaging (MRI), x-ray, ultrasound image data set, or from a set of points collected on the bone intra-operatively. A set of 3-D computer aided design (CAD) models of the manufacturer's prosthesis are pre-loaded in the software that allows the user to place the components of a desired prosthesis to the 3-D model of the boney anatomy to designate the best fit, position, and orientation of the implant to the bone.

As used herein, the term “real-time” refers to the processing of input data within milliseconds such that calculated values are available within 2 seconds of computational initiation.

As used herein, a “cut-file” refers to a software file having a set of instructions to automatically or haptically control a surgical robot. The set of instructions illustratively include cut paths, points, virtual boundaries, velocities, accelerations, spindle speeds, feed rates, and any combination thereof to automatically or haptically control the robot. One or more cut-files may be generated based on the geometry of the implant, the geometry of the bone models, a planned position of the implant models relative to the bone models, or a combination thereof using computer-aided manufacturing (CAM) techniques.

As used herein, the term “registration” refers to: the determination of the spatial relationship between two or more objects; the determining of a coordinate transformation between two or more coordinate systems associated with those objects; and/or the mapping of an object onto another object. Examples of objects routinely registered in an operating room (OR) illustratively include: computer-assisted systems/devices; anatomy (e.g., bone); pre-procedure data (e.g., 3-D virtual bone models); medical planning data (e.g., an implant model positioned relative to pre-operative bone data, a cut-file defined relative to an implant model and/or pre-operative bone data, virtual boundaries defined relative to an implant model and/or pre-operative bone data, virtual planes defined relative to an implant model and/or pre-operative bone data, or other cutting parameters associated with or defined relative to an implant model and/or the pre-operative bone data); and any external landmarks (e.g., a tracking array affixed to a bone, an anatomical landmark, a designated point/feature on a bone, etc.) associated with the tissue (if such landmarks exist). Methods of registration known in the art are described in U.S. Pat. Nos. 6,033,415; 8,010,177; 8,036,441; and 8,287,522; and U.S. Patent Application Publication 2016/0338776, which patents and publications are hereby incorporated herein by reference. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a representation of the bone (e.g., a 3-D bone model). The points collected from the surface of a bone with the digitizer may be matched using iterative closest point (ICP) algorithms to generate a transformation matrix. The transformation matrix provides the correspondence between the position of the bone in an operating room (OR) with the bone model to permit the surgical device to execute the plan.

Also used herein is the term “optical communication” which refers to wireless data transfer via infrared or visible light that are illustratively described in U.S. Pat. No. 10,507,063 and assigned to the assignee of the present application.

Embodiments of the invention provide for the alignment of a keel punch in a planned position and orientation to form one or more keel receiving features in a bone. In the current surgical approach, knowledge is required as to the location of the patient's bones relative to each other as well as the surgical plan. If the surgical plan registered to the bones does not allow for the keel receiving features to be cut with the current patient position, the surgeon must un-fix the bones from the surgical robot, adjust the patient position, and then recover the registration prior to completion. Embodiments of the present invention removes this constraint and allows the surgeon to reposition the patient prior to punching the keel receiving features without losing the alignment. Additionally, this approach protects the robot from the forces required to punch the keel features manually, which would otherwise occur if the surgical robot were to fixedly hold the keel punching tool in place while punching the keel features. Finally, embodiments of the invention do not require a tool change, or require the end-effector to be fixedly attached to any of the components while aligning the keel punch alignment guide on the bone.

Embodiments of the inventive method provide for the alignment of a keel punch alignment guide on a tibial bone with the aid of a surgical robot. After the tibial plateau is resurfaced and the tibial post hole is formed by a surgical robot, the surgical robot moves to an alignment position. The user is instructed to place a keel punch alignment guide on the tibial plateau, and assemble the keel punch alignment guide onto a distal portion of the end-effector. This provides medial-lateral and axial alignment. Furthermore, the surgical system instructs the user to insert a temporary keel post tool through the keel punch alignment guide and into the tibial post hole cut by the robot. The keel punch alignment guide is now fully aligned in at least three degrees-of-freedom to guide a keel punch to form the keel receiving features in the planned POSE. Next, the user is instructed to affix the alignment guide to the tibial plateau using small alignment tacks. The surgeon is asked to confirm the fixation and upon confirmation the end-effector is moved away from the alignment position and the temporary keel post tool is removed from the keel punch alignment guide. The user then aligns the keel punch with one or more guiding apertures in the keel punch alignment guide, and manually punches the keel receiving features into the bone. Because the surgical robot is no longer part of the procedure, the position of the tibia no longer needs to be fixed to the robot, which allows the surgeon to punch the keel features without concern for bone motion. Finally, the user removes the keel punch alignment guide from the bone and implants the tibial implant component.

It should be appreciated that while the methods described herein make reference to the preparation of a tibia during total knee arthroplasty, embodiments of the present invention may be applied or adapted for other bones and orthopedic surgical procedures illustratively including total hip arthroplasty, hip resurfacing, unicondylar knee arthroplasty, ankle arthroplasty, shoulder arthroplasty, bone plate fixation, and other orthopedic procedures as detailed with respect to the aforementioned anatomical joints and/or bones that include, for example, vertebrae, long bones, skull, jaw, metacarpals, or metatarsals. Unless otherwise specified, the components detailed herein are formed of conventional surgical materials illustratively including sterilizable metals, polymers, intermetallics, ceramics, and composite materials. Stainless steel is exemplary of such surgical materials.

Furthermore, it should be appreciated that while the system and methods described herein make reference to implant preparation/guide tools for a keel punch, embodiments of the present invention may be applied or adapted for other implant preparation/guide tools. For example, the robot may be configured to align cut guides, fracture plates, templates, guide tubes, etc. relative to the bone in order to assist a user in preparing the bone to receive an implant.

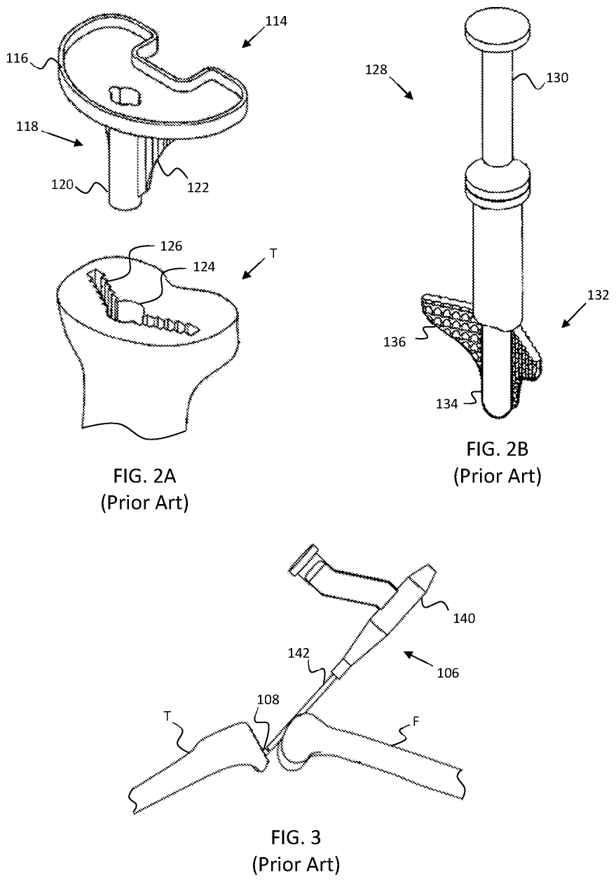

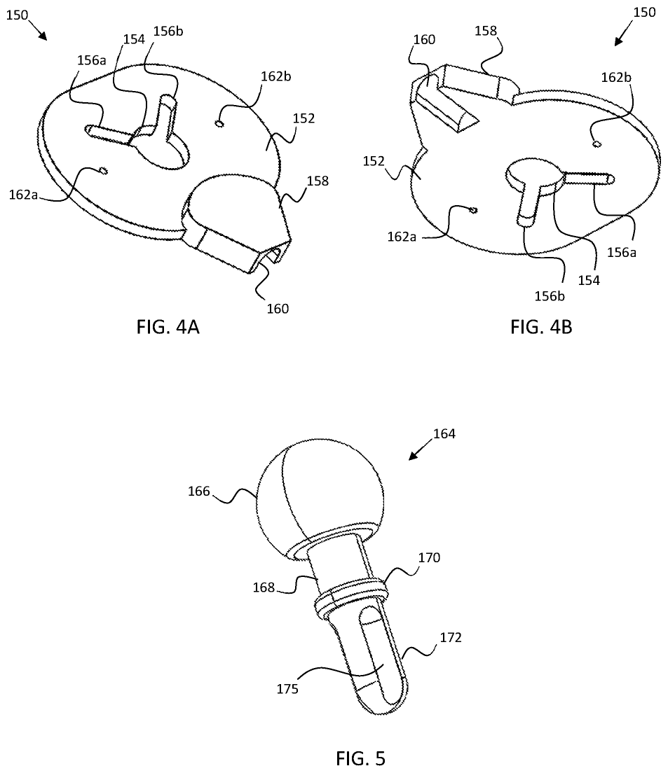

With reference now to the figures, FIGS. 4A and 4B depict a keel punch alignment guide 150 where FIG. 4A is a top perspective view thereof, and FIG. 4B is a bottom perspective view thereof. The keel punch alignment guide 150 is configured to guide a keel punch to form one or more keel receiving features in the bone in a planned position and orientation. The keel punch alignment guide 150 may generally be in the form of a plate 152 having one or more guiding apertures that guide the keel punch therethrough. The plate 152 may be planar in shape such that one side of the plate 152 may lie or rest on a resurfaced planar surface of a bone, such as the resurfaced tibial plateau in TKA. The one or more guiding apertures generally have a shape and size that matches, or slightly exceeds, the outline or perimeter of the keel punch such that the keel punch can travel through the one or more guiding apertures. For example, the one or more guiding apertures may include in certain inventive embodiments, in certain inventive embodiments, a post guiding aperture 154 and groove guiding apertures (156a, 156b) for guiding the keel post punch component 134 and keel groove punch components 136, respectively, of the keel punch 132 shown in FIG. 2B. In this instance, the post guiding aperture 154 has a circular shape with a diameter equal to or slightly exceeding the diameter of the post punch component 134, and the groove guiding apertures (156a, 156b) are in the shape of slots having a width equal to or slightly exceeding the width of the keel groove punch components 136. The shape and size of the one or more guiding apertures may slightly exceed the outline or perimeter of the keel punch by 1% to 5% of the maximum outline or perimeter of the keel punch to ensure the keel punch can travel through the one or more guiding apertures without affecting accuracy. It should be appreciated that various keel punch alignment guides 150 may be designed and manufactured to accommodate different implant families, implant lines, and/or implant sizes.

The keel punch alignment guide 150 further includes an end-effector interaction member (EIM) 158. The EIM 158 is configured to interact with an end-effector of a surgical robot to permit the end-effector to assist in aligning the keel punch alignment guide 150. The EIM 158 may be part of the plate 152, or project from the plate 152, and have a semi-circular channel 160 that assembles with an interacting portion of the end-effector. The interacting portion of the end-effector may include in certain inventive embodiments, in certain inventive embodiments: the end-effector tool tip, the end-effector tool tip and a distal portion of a shaft of the tool; or the end-effector tool tip and a distal portion of a sleeve that surrounds a shaft of the tool. In particular embodiments, the EIM 158 and end-effector assemble together by simply resting against each other such that the end-effector is never fixedly attached to the keel punch alignment guide 150. For example, the semi-circular channel 160 may simply capture the interacting portion of the end-effector therein without the use of any securing or attachment mechanisms. This allows the surgical robot to quickly align the keel punch alignment guide 150 and be removed therefrom once aligned. The radius of the semi-circular channel 160 may therefore have a radius that slightly exceeds (e.g., 1% to 5% larger than) the interaction portion of the end-effector (e.g., 1% larger than the radius of the end-effector tool tip). It should be appreciated that other shapes or forms of an EIM 158 are possible to quickly assemble, disassemble, or align with the interaction portion of the end-effector. These other shape or forms may illustratively include an enclosed channel, receptacle, a notch, a divot, a hole, or a groove. It is further contemplated that the EIM 158 may be a marking (e.g., an arrow or line) that a user can align with a longitudinal axis of the end-effector. If a marking is used, there may be no need for physical contact between the EIM 158 and the interacting portion of the end-effector. In this case, the interacting portion of the end-effector is the longitudinal axis of the end-effector.

With reference now to FIG. 5, a temporary keel post tool 164 is shown generally at 164. The temporary keel post tool 164 is configured to be temporarily inserted into a prepared post hole formed in the bone to assist with aligning the keel punch alignment guide 150 in at least one degree-of-freedom. The temporary keel post tool 164 may include in certain inventive embodiments, in certain inventive embodiments, a handle 166, a shaft 168, a collar 170, and an insertable post 172. The handle 166 is disposed at a proximal end of the shaft 168, and the insertable post 172 is disposed at a distal end of the shaft 168. The collar 170 is positioned between the handle 166 and the insertable post 172, and may be positioned proximally adjacent to the insertable post 172. The handle 166 is configured to be held by a user may have the general shape of a sphere. The collar 170 is configured to abut against a top surface of the keel punch alignment. The collar 170 may have the shape of a cylinder or ring with a diameter that exceeds the diameter or width of the insertable post 172. The insertable post 172 is configured to be inserted in a prepared keel post hole formed in the bone, and may have the general shape of a cylinder with a hemispherical distal end. The insertable post 172 may further have notches or grooves 175 formed along its length.

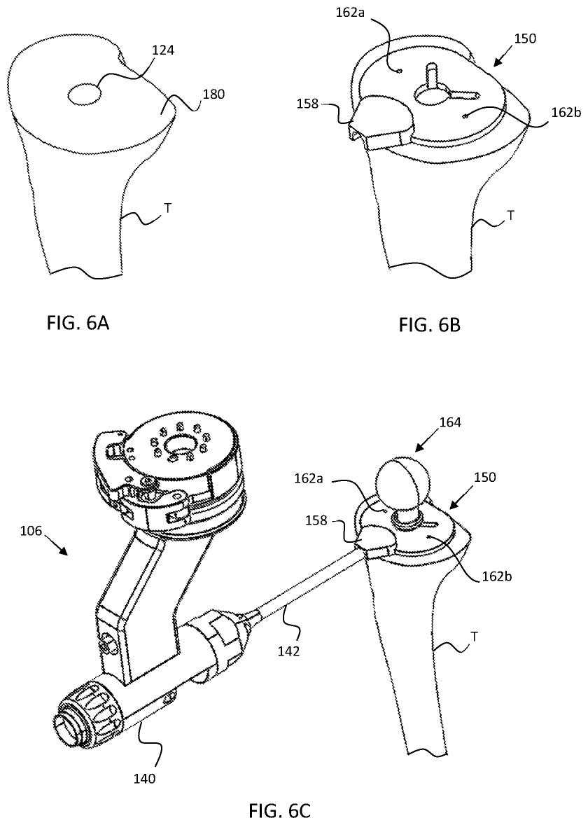

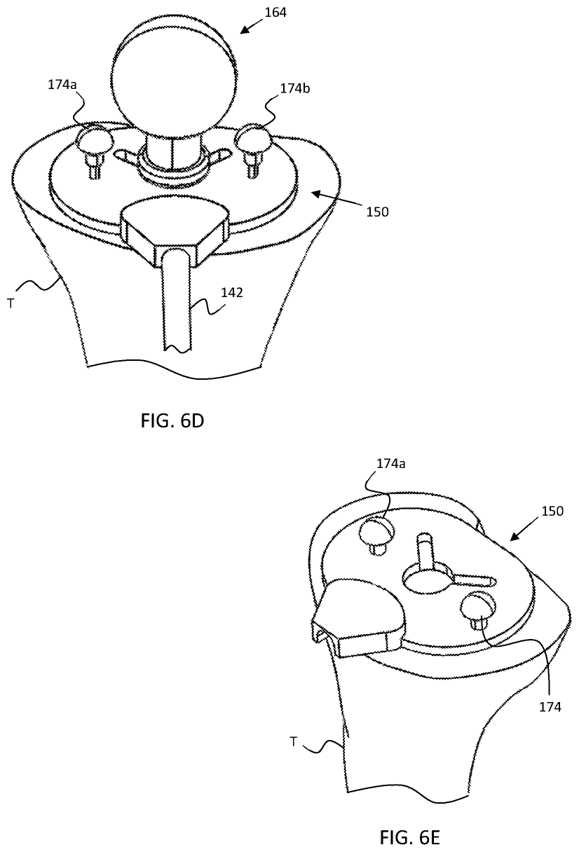

With reference to FIGS. 6A to 6E, embodiments of an inventive method to align a keel punch alignment guide 150 on a tibial bone are illustrated pictorially. Prior to the inventive procedure, a surgical plan is generated using a pre-operative planning software program. The pre-operative software program contains tools for a user to position one or more implant components (in the form of CAD models) relative to three-dimensional (3-D) models of the patient's bones to designate the best fit, fill, or alignment for the final implants on the bone. An exemplary best fit methodology is detailed in “Improving Accuracy in Knee Arthroplasty” by Thienpont Emmanuel, Jaypee Brothers Medical Publishers Pvt. Ltd., Dec. 15, 2012 with particular reference to pages 266-277. The final surgical plan includes instructions (e.g., a cut-file) for a surgical robot 100 to mill the bone to receive the implants as defined in the plan. In the operating room (OR), the surgical plan is registered to the bone and the surgical robot 100 proceeds to prepare the bones. As shown in FIG. 6A, the surgical robot 100 may prepare the tibia T to form a resurfaced tibial plateau 180 and a keel post hole 124, while the remaining keel receiving features (e.g., grooves 126 as shown in FIG. 2A) will be prepared with the use of the keel punch alignment guide 150 and a keel punching tool 128 (as shown in FIG. 2B) as further described below. A user may decide to forego the robotic preparation of the remaining keel receiving features in favor of using a keel punching tool 128 either pre-operatively or intra-operatively. If the decision is made pre-operatively, the instructions for the surgical robot 100 to mill the remaining keel receiving features may be excluded from the surgical plan. If the decision is made intra-operatively, a user may have the option to skip this milling step in the surgical workflow. Alternatively, the surgical robot 100 may, by default, lack the instructions to mill the remaining keel receiving features, and/or this milling step may be automatically skipped.

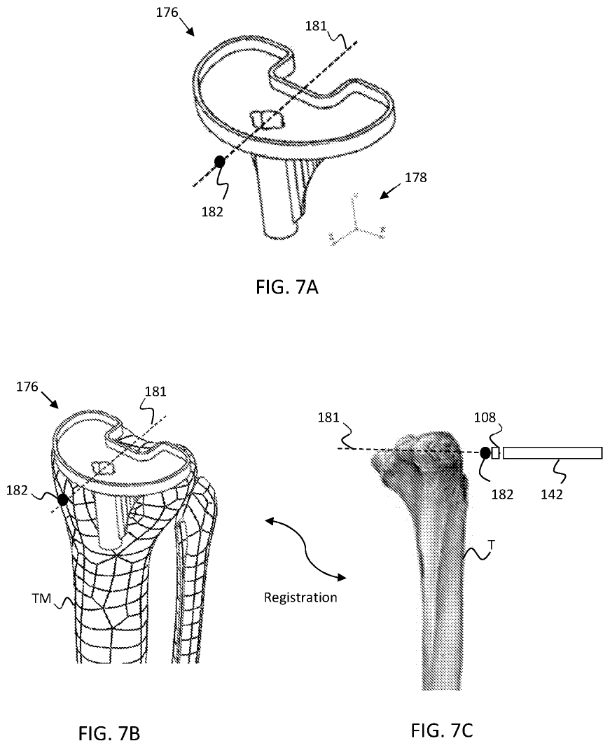

After the surgical robot 100 resurfaces the tibial plateau and mills the keel post hole 124, the surgical system may instruct the user to remove any remaining bone proximal to the resurfaced tibial plateau 180 manually if any remaining bone exists. The keel punch alignment guide 150 is now ready to be aligned on the resurfaced tibial plateau 180 as shown in FIG. 6B. The surgical robot 100 is instructed to move the end-effector 106 to an alignment position. The alignment position for the end-effector 106 may be defined based on the geometry of the keel punch alignment guide 150, the geometry of the tibial implant component, and/or the planned POSE for the tibial implant component relative to the bone. A method to define the alignment position for the end-effector 106 is described below with reference to FIGS. 7A and 7B. The surgical system then instructs the user to place the keel punch alignment guide 150 on the resurfaced tibial plateau 180 and align or assemble the EIM 158 with the interacting portion of the end-effector 106. The surgical system may further instruct the user to insert the temporary keel post tool 164 through the keel punch alignment guide 150 and into the keel post hole 124. More specifically, the insertable post 172 of the keel post tool 164 may be inserted through the post guiding aperture 154 and into the keel post hole 124, where the collar 170 of the tool 164 abuts against and holds the keel punch alignment guide 150 on the resurfaced tibial plateau 180. FIG. 6C depicts: a) the EIM 158 capturing the end-effector tool tip 108 and distal portion of the end-effector sleeve 142; and b) the temporary keel post tool 164 inserted through the post guiding aperture 154 and into the keel post hole 124. The keel punch alignment guide 150 is now aligned in at least three degrees-of-freedom to guide a keel punching tool 128 to form the keel receiving feature in the planned POSE. The temporary keel post tool 164 locks in medial-lateral and anterior-posterior translation, the resurfaced tibial plateau 180 locks in proximal-distal translation, varus-valgus rotation, and flexion-extension rotation, and the interaction portion of the end-effector 106 locks in internal-external rotation, and may further lock in medial-lateral translation and anterior-posterior translation. After the keel punch alignment guide 150 is aligned on the resurfaced tibial plateau 180, a pair of tacks (174a, 174b) are used to secure the keel punch alignment guide 150 on the bone as shown in FIG. 6D. Each tack (174a or 174b) is inserted through a hole (162a or 162b) as shown in FIGS. 4A and 4B in the keel punch alignment guide 150 and into the underlying bone. Once the keel punch alignment guide 150 is secured, the end-effector 106 is moved away from the alignment position and the temporary keel post tool 164 is removed from the keel punch alignment guide 150 to leave the keel punch alignment guide 150 on the resurfaced tibial plateau 180 as shown in FIG. 6E. At this point, the tibia T may move, or be moved, to any position without losing the alignment of the keel punch alignment guide 150. The user may further un-fix the tibial bone from the surgical robot if needed since the surgical robot is no longer needed for the tibial portion of the procedure. The user may then form the keel receiving features in the bone with a keel punching tool 128 by punching the keel punch 132 through the one or more guiding apertures (154, 156) in the keel punch alignment guide 150 and into the bone. The keel punch alignment guide 150 is then removed from the bone, and the user implants the tibial implant component into the bone to complete the tibial portion of the procedure.

In a specific embodiment, a haptically controlled surgical robot may be used. The keel punch alignment guide 150 may be attachable to the end-effector 106. After the user prepares the bone with the surgical robot, the user attaches the keel punch alignment guide 150 to the end-effector 106. The user then wields the end-effector 106 in the vicinity of the planned position for the keel punch 128. The surgical robot then haptically constrains or guides the end-effector 106 such that the keel punch alignment guide 150 is aligned in the pre-planned position and orientation. Once aligned, the user may assemble the tacks (162a, 162b) to secure the alignment guide 150 to the bone and then create the keel receiving features by guiding a keel punch 128 through the one or more guiding apertures (154, 156) in the alignment guide 150.

### End-Effector Alignment Position for Aligning the Keel Punch Alignment Guide

The alignment position for the end-effector 106 may be defined by several different methods, which will be apparent to those skilled in the art. FIGS. 7A-7C depict at least one method for defining the end-effector alignment position. FIG. 7A depicts a 3-D tibial implant model 176. The implant model 176 may be generated from the manufacturer, designed by a user, or otherwise generated based on the dimensions of a physical tibial implant. The implant model 176 may further have a coordinate system 178 to define coordinates, axes, or planes on the implant model 176 relative to the coordinate system 178. The end-effector alignment position may be defined as an axis 181 and a point 182, which may be defined relative to the tibial implant model 176. The axis 181 and the point 182 may be user-defined in the pre-operative software program, defined by the implant manufacturer, or defined by the robot manufacturer. The POSE of the axis 181 is defined to permit the end-effector to align the keel punch alignment guide 150 in a planned internal-external rotation, or axial alignment. In a particular embodiment, the axis is defined as being: a) coincident with the anterior-posterior axis of the tibial implant component; b) equidistant between the medial and lateral sides of the tibial implant component; and c) proximally/distally aligned in a superior-inferior direction of the tibial component (e.g., aligned with the inferior surface of the tibial base plate). The proximal/distal POSE of the axis 181 may also be defined to be in-plane with the resurfaced tibial plateau 180 as defined in the surgical plan, and may further be offset therefrom by the geometry of the EIM 158 of the keel punch alignment guide 150 such that the EIM 158 can interact with the end-effector 106 as described above. The point 182 may be defined along the axis 181 and at a set-distance anterior to the tibial implant 176. The set-distance may be chosen to ensure the end-effector 106 does not contact the bone in the OR for safety purposes. In a particular embodiment, the set-distance is based on the projection distance of the EIM 158, or the length of the EIM channel 160, such that the EIM 158 can capture the interaction portion of the end-effector 106. The POSE of the axis 181 and the point 182 is then translated into the operating room by the following. FIG. 7B depicts the tibial implant model 176 in a planned POSE relative to a 3-D tibial bone model TM. Once the planned POSE of the implant model 176 is finalized, the POSE of the implant model 176, the axis 181, and the point 182 are fixed relative to the 3-D bone model TM. As shown in FIG. 7C, in the OR, the 3-D bone model is registered to the tibia T in the coordinate system of the surgical robot 100. The registration provides the surgical robot 100 with the POSE of the axis 181 and the point 182 in physical space relative to the tibia T. The surgical robot 100 can then move the end-effector 106 to the alignment position by orienting the longitudinal axis of the end-effector 106 to be coincident with the axis 181 and positioning the end-effector tool tip 108 at the point 182. The user may then assemble the EIM 158 with the interaction portion of the end-effector 106 to align the keel punch alignment guide 150 on the bone as described above.

### Robotic Surgical System

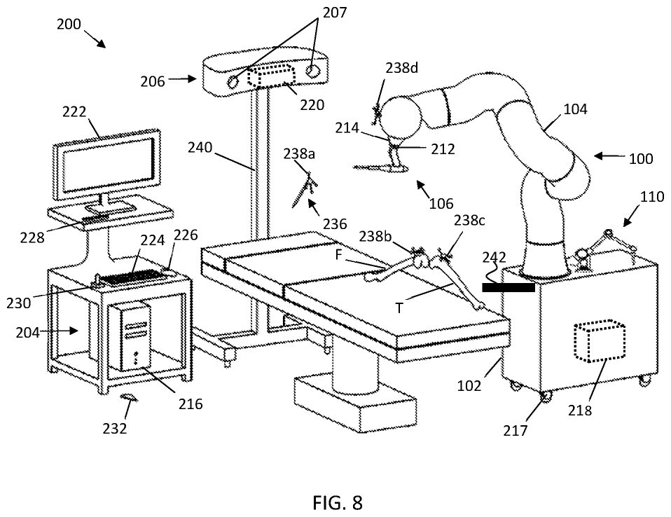

FIG. 8 depicts a robotic surgical system 200 in the context of an operating room (OR) to prepare a femoral and tibial bone for total knee arthroplasty and assist with aligning a keel punch alignment guide 150 on a bone as described above. The surgical system 200 includes a surgical robot 100, a computing system 204, and an optional tracking system 206. The surgical robot 100 may include in certain inventive embodiments, in certain inventive embodiments, a movable base 102, a manipulator arm 104 connected to the base 102, an end-effector 106 located at a distal end 212 of the manipulator arm 104, and a force sensor 214 positioned proximal to the end-effector 106 for sensing forces experienced on the end-effector 106. The base 102 includes a set of wheels 217 to maneuver the base 102, which may be fixed into position using a braking mechanism such as a hydraulic brake. The base 102 may further include an actuator to adjust the height of the manipulator arm 104. The manipulator arm 104 includes various joints, links, and sensors (e.g., encoders) to accurately manipulate the end-effector 106 in various degrees of freedom. The joints are illustratively prismatic, revolute, spherical, or a combination thereof. The end-effector 106 may be a motor-driven end-mill, cutter, drill-bit, or other bone removal device.

The computing system 204 may generally include a planning computer 216; a device computer 218; a tracking computer 220 (if present); and peripheral devices. The planning computer 216, device computer 218, and tracking computer 220 may be separate entities, one-in-the-same, or combinations thereof depending on the surgical system. Further, in some embodiments, a combination of the planning computer 216, the device computer 218, and/or tracking computer 220 are connected via a wired or wireless communication. The peripheral devices allow a user to interface with the surgical system components and may include in certain inventive embodiments, in certain inventive embodiments: one or more user-interfaces, such as a display or monitor 222 to display a graphical user interface (GUI); and user-input mechanisms, such as a keyboard 224, mouse 226, pendent 228, joystick 230, foot pedal 232, or the monitor 222 in some inventive embodiments has touchscreen capabilities.

The planning computer 216 contains hardware (e.g., processors, controllers, and/or memory), software, data and/or utilities that are in some inventive embodiments dedicated to the planning of a surgical procedure, either pre-operatively or intra-operatively. This may include in certain inventive embodiments, reading pre-operative bone data, displaying pre-operative bone data, manipulating pre-operative bone data (e.g., image segmentation), constructing three-dimensional (3D) virtual models, storing computer-aided design (CAD) files, providing various tools, functions, or widgets to aid a user in planning the surgical procedure, and generating surgical plan data. The final surgical plan may include in certain inventive embodiments, pre-operative bone data, patient data, registration data including the position of a set of points P defined relative to the pre-operative bone data for registration, trajectory parameters (e.g., axis 181 and point 182), and/or a set of instructions to operate the surgical robot 100. The set of instructions may include in certain inventive embodiments, instructions for the surgical robot to modify a volume of bone to receive an implant. The set of instructions may illustratively be: a cut-file having a set of cutting parameters/instructions (e.g., cut paths, velocities) to automatically modify the volume of bone; a set of virtual boundaries defined to haptically constrain a tool within the defined boundaries to modify the bone; a set of boundaries coupled with power or actuation control of a tracked surgical device to ensure the end-effector only removes bone within the boundaries; a set of planes or drill holes to drill pins or tunnels in the bone; or a graphically navigated set of instructions for modifying the tissue. In particular embodiments, the set of instructions is a cut-file for execution by a surgical robot to automatically modify the volume of bone, which is advantageous from an accuracy and usability perspective. The surgical plan data generated from the planning computer 216 may be transferred to the device computer 218 and/or tracking computer 220 through a wired or wireless connection in the operating room (OR); or transferred via a non-transient data storage medium (e.g., a compact disc (CD), a portable universal serial bus (USB) drive) if the planning computer 216 is located outside the OR.

The device computer 218 in some inventive embodiments is housed in the moveable base 102 and contains hardware, software, data and/or utilities that are preferably dedicated to the operation of the surgical robotic device 100. This may include in certain inventive embodiments, surgical device control, robotic manipulator control, the processing of kinematic and inverse kinematic data, the execution of registration algorithms, the execution of calibration routines, the execution of the set of instructions (e.g., cut-files, the trajectory parameters), coordinate transformation processing, providing workflow instructions to a user, and utilizing position and orientation (POSE) data from the tracking system 206 if a tracking system is present. In some embodiments, the surgical system 200 includes a mechanical digitizer arm 110 attached to the base 102. The digitizer arm 110 may have its own computer or may be directly connected with the device computer 218. In other inventive embodiments, the system includes a tracked hand-held digitizer device 236 with a probe tip to be tracked by the tracking system 206.

The tracking system 206 may be an optical tracking system that includes two or more optical receivers 207 to detect the position of fiducial markers (e.g., retroreflective spheres, active light emitting diodes (LEDs)) uniquely arranged on rigid bodies. The fiducial markers arranged on a rigid body are collectively referred to as a tracking array (238a, 238b, 238c, 238d), where each tracking array has a unique arrangement of fiducial markers, or a unique transmitting wavelength/frequency if the markers are active LEDs. An example of an optical tracking system is described in U.S. Pat. No. 6,061,644. The tracking system 206 may be built into a surgical light, located on a boom, a stand 240, or built into the walls or ceilings of the OR. The tracking system computer 220 may include in certain inventive embodiments, tracking hardware, software, data, and/or utilities to determine the POSE of objects (e.g., bones B, surgical robotic device 100) in a local or global coordinate frame. The POSE of the objects is collectively referred to herein as POSE data, where this POSE data may be communicated to the device computer 218 through a wired or wireless connection. Alternatively, the device computer 218 may determine the POSE data using the position of the fiducial markers detected from the optical receivers 207 directly.

The POSE data is determined using the position data detected from the optical receivers 207 and operations/processes such as image processing, image filtering, triangulation algorithms, geometric relationship processing, registration algorithms, calibration algorithms, and coordinate transformation processing.

The POSE data is used by the computing system 204 during the procedure to update the POSE and/or coordinate transforms of the bone B, the surgical plan, and the surgical robot 100 as the manipulator arm 104 and/or bone(s) (F, T) move during the procedure, such that the surgical robot 100 can accurately execute the surgical plan.

In specific embodiments, the surgical system 200 does not include a tracking system 206, but instead employs a mechanical digitizer arm 110, a bone fixation system having bone fixation hardware 242 to fix the bone relative to the surgical robot 100, and a bone motion monitoring system to monitor bone movement, all of which are described in U.S. Pat. No. 5,086,401.

The device computer 218 further includes one or more processors, and non-transient memory having software executable instructions stored therein for performing embodiments of the inventive methods described herein. More particularly, the software executable instructions when executed by the processor performs the following: registers the surgical plan relative to the bone; controls a surgical robot to resurface the tibial plateau and mill a keel post hole in the bone; moves an end-effector into an alignment position; instruct a user, by way of a GUI, to assemble a keel punch alignment guide on the bone, and assemble the keel punch alignment guide with an interaction portion of the end-effector; instruct a user, by way of GUI, to insert a temporary keel post tool through the keel punch alignment guide and into the keel post hole; instruct a user to secure a keel punch alignment guide to the bone when the keel punch alignment guide is aligned on the bone; move the end-effector away from keel punch alignment guide once the keel punch alignment guide is secured to the bone; and instruct a user to form one or more keel receiving features in the bone by guiding a keel punch through one or more guiding apertures in the keel punch alignment guide.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A system for aligning a keel punch in a planned position and orientation, the system comprising:
a keel punch alignment guide configured to guide the keel punch to form one or more keel receiving features in a material; and
a keel post tool configured to be temporarily inserted into a prepared post hole formed in the material to assist with aligning the keel punch alignment guide.

2. The system of claim 1 wherein the keel punch alignment guide has a planar shape.

3. The system of claim 1 wherein the keel punch alignment guide further comprises one or more guiding apertures.

4. The system of claim 3 wherein one or more guiding apertures have a shape and size that matches, or slightly exceeds, the outline or perimeter of the keel punch.

5. The system of claim 4 wherein the shape and size of the one or more guiding apertures exceeds the outline or perimeter of the keel punch by from 1% to 5% of the maximum outline or perimeter of the keel punch.

6. The system of claim 3 wherein one or more guiding apertures include a post guiding aperture and a set of groove guiding apertures that correspond to a keel post punch component and a set of keel groove punch components, respectively, of the keel punch.

7. The system of claim 1 wherein the keel punch alignment guide further comprises an end-effector interaction member configured to interact with an end-effector of a surgical robot to permit the end-effector to assist in aligning the keel punch alignment guide and the material is a bone of a subject.

8. The system of claim 7 wherein the end-effector interaction member further comprises a semi-circular channel that assembles with an interacting portion of the end-effector.

9. The system of claim 8 wherein the interacting portion of the end-effector is one of: an end-effector tool tip, the end-effector tool tip and a distal portion of a shaft of a tool, or the end-effector tool tip and a distal portion of a sleeve that surrounds a shaft of the tool.

10. The system of claim 8 wherein the semi-circular channel has a radius that is from 1% to 5% larger than a second radius of the interaction portion of the end-effector.

11. The system of claim 7 wherein the end-effector interaction member is at least one of an enclosed channel, a receptacle, a notch, a divot, a hole, a groove, or a marking that aligns with a longitudinal axis of the end-effector.

12. The system of claim 1 further comprising at least one securement configured to be inserted through apertures in the keel punch alignment guide and into an underlying bone of a subject.

13. The system of claim 1 wherein the keel post tool comprises a handle, a shaft, a collar, and an insertable post.

14. The system of claim 13 wherein the handle is shaped as a sphere.

15. The system of claim 13 wherein the collar is configured to abut against a top surface of the keel punch alignment guide or the collar has a collar diameter that exceeds the diameter or width of the insertable post.

16. (canceled)

17. The system of claim 13 wherein the insertable post has a post length and further comprises notches or grooves formed along the post length.

18. A method for aligning a keel punch in a planned position and orientation relative to a bone of a subject to form one or more keel receiving features in the bone during a computer-assisted surgical procedure, the method comprising:
positioning an end-effector of a surgical robot to an alignment position based on at least one of a geometry of a keel punch alignment guide, a geometry of an implant component, and a planned position and orientation (POSE) for the implant component relative to the bone;
placing a keel punch alignment guide on a prepared bone surface of the bone;
aligning an end-effector interaction member on the keel punch alignment guide with the end-effector of the surgical robot; and
securing the aligned keel punch alignment guide to the bone; and
removing the end-effector.

19. The method of claim 18 further comprising preparing the prepared bone surface by milling a flat bone surface and creating a keel post hole in the bone.

20. The method of claim 18 further comprising inserting a temporary keel post tool through a post guiding aperture in the keel punch alignment guide and into a keel post hole created in the bone.

21. The method of claim 18 wherein the surgical procedure is a total knee arthroplasty (TKA) and the bone is a tibia or a femur.
